(19) United States
(12) Patent Application Publication
(10) Pub. No.: US 2025/0285264 A1
(43) Pub. Date: Sep. 11, 2025

(54) METHOD AND DEVICE FOR PROCESSING THREE-DIMENSIONAL ORAL CAVITY MODEL

(71) Applicant: MEDIT CORP., Seoul (KR)

(72) Inventors: Dong Hoon LEE, Seoul (KR); Soo Bok LEE, Seoul (KR)

(73) Assignee: MEDIT CORP., Seoul (KR)

(21) Appl. No.: 18/274,150

(22) PCT Filed: Jan. 25, 2022
(86) PCT No.: PCT/KR2022/001310

§ 371 Date: Sep. 19, 2024

(30) Foreign Application Priority Data
Jan. 26, 2021 (KR) 10-2021-0010847

(51) Int. Cl. G06T 7/00

(52) U.S. Cl. CPC G06T 7/0012; G06T 2207/10024; G06T 2207/10028; G06T 2207/30036

(57) ABSTRACT

Disclosed are a method and device for processing a three-dimensional intraoral model. The disclosed method of processing a three-dimensional intraoral model includes displaying a first three-dimensional intraoral model on a display, receiving an input for setting at least a part of the displayed first three-dimensional intraoral model as an attribute control area, obtaining a second three-dimensional intraoral model, changing a color of the at least a part of the first three-dimensional intraoral model set as the attribute control area, to a target color representing a color of an area of the obtained second three-dimensional intraoral model corresponding to the attribute control area, and displaying, on the display, the first three-dimensional intraoral model in which the color of the attribute control area is changed to the target color.

### TECHNICAL FIELD

The disclosed embodiment relates to a method and device for processing a three-dimensional intraoral model. In particularly, the disclosed embodiment relates to a method and device for processing a three-dimensional intraoral model for controlling a color of at least a part of the three-dimensional intraoral model.

### BACKGROUND ART

Recently, processes of producing a dental prosthesis and performing orthodontic treatment has been digitized to increase work efficiency and shorten work time in dental laboratories.

In particular, a prosthetic or orthodontic plan may established more conveniently by, for obtaining information about the oral cavity of a patient, inserting an intraoral scanner into the oral cavity of a patient, obtaining intraoral information of the patient, and generating a three-dimensional intraoral model based on the intraoral information. As such, when digitally establishing a prosthetic or orthodontic plan, generation of a three-dimensional intraoral model is an important process, and there is a need to control or change at least some attributes, particularly the color, of a generated three-dimensional intraoral model for various purposes.

### DESCRIPTION OF EMBODIMENTS

### Technical Problem

The disclosed embodiment provides a method and device for processing a three-dimensional intraoral model for controlling an attribute of at least a partial area set in the three-dimensional intraoral model.

### Solution to Problem

A method of processing a three-dimensional intraoral model according to an embodiment includes displaying a first three-dimensional intraoral model on a display, receiving an input for setting at least a part of the displayed first three-dimensional intraoral model as an attribute control area, obtaining a second three-dimensional intraoral model, changing a color of the at least a part of the first three-dimensional intraoral model set as the attribute control area, to a target color representing a color of an area of the obtained second three-dimensional intraoral model corresponding to the attribute control area, and displaying, on the display, the first three-dimensional intraoral model in which the color of the attribute control area is changed to the target color.

According to an embodiment, the changing to the target color may include identifying the area of the obtained second three-dimensional intraoral model corresponding to the attribute control area, and obtaining the target color by extracting color information from the identified area.

According to an embodiment, the obtaining of the second three-dimensional intraoral model may include obtaining the second three-dimensional intraoral model based on data obtained by scanning a tooth in a state in which an object is arranged to be overlaid on a front surface of the tooth.

According to an embodiment, the object may be arranged at a tooth position corresponding to a position set as the attribute control area in the three-dimensional intraoral model.

According to an embodiment, the obtaining of the target color may include identifying a position set as the attribute control area in the first three-dimensional intraoral model, and obtaining the target color by extracting color information from an area of the second three-dimensional intraoral model corresponding to the identified position.

According to an embodiment, the receiving of the input for setting the at least a part of the displayed first three-dimensional intraoral model as the attribute control area may include receiving an input for setting the attribute control area in the first three-dimensional intraoral model obtained by scanning a tooth on which cord packing has been performed.

According to an embodiment, the changing of the color of the at least a part of the first three-dimensional intraoral model set as the attribute control area to the target color may include obtaining the second three-dimensional intraoral model generated by scanning a tooth from which foreign substances are removed, obtaining the target color by extracting color information from the area of the obtained second three-dimensional intraoral model corresponding to the attribute control area, and changing the color of the at least a part of the first three-dimensional intraoral model set as the attribute control area to the obtained target color.

According to an embodiment, the first three-dimensional intraoral model may include a three-dimensional intraoral model obtained by scanning a dental impression.

According to an embodiment, the changing of the color of the at least a part of the first three-dimensional intraoral model set as the attribute control area to the target color may include obtaining the second three-dimensional intraoral model by scanning a tooth, obtaining the target color by extracting color information from the area of the obtained second three-dimensional intraoral model corresponding to the attribute control area, and changing the color of the at least a part of the first three-dimensional intraoral model set as the attribute control area to the obtained target color.

A device for processing a three-dimensional intraoral model according to an embodiment includes a processor and a memory, wherein the processor is configured to execute one or more instructions stored in the memory to display a first three-dimensional intraoral model on a display, receive an input for setting at least a part of the displayed first three-dimensional intraoral model as an attribute control area, obtain a second three-dimensional intraoral model, change a color of the at least a part of the first three-dimensional intraoral model set as the attribute control area, to a target color representing a color of an area of the obtained second three-dimensional intraoral model corresponding to the attribute control area, and display, on the display, the first three-dimensional intraoral model in which the color of the attribute control area is changed to the target color.

### Advantageous Effects of Disclosure

According to a method and device for processing a three-dimensional intraoral model according to the disclosed embodiment, a desired three-dimensional intraoral model may be obtained simply and conveniently by setting a partial area of a generated three-dimensional intraoral model as an attribute control area, and changing only an attribute of the set attribute control area.

### BRIEF DESCRIPTION OF DRAWINGS

The present disclosure may be readily understood with a combination of the following detailed descriptions and the accompanying drawings, wherein reference numbers refer to structural elements.

FIG. 1 is a diagram for describing a digital intraoral model processing system according to the disclosed embodiment.

FIG. 2 is a block diagram illustrating a data processing device according to an embodiment.

FIG. 3 is a flowchart illustrating a method, performed by a data processing device, of processing a three-dimensional intraoral model, according to the disclosed embodiment.

FIG. 4 is a diagram illustrating an example of a first three-dimensional intraoral model obtained by the data processing device, according to an embodiment.

FIG. 5 is a flowchart of an example of a method of obtaining a target color for controlling an attribute of an attribute control area set in a part of a first three-dimensional intraoral model, according to an embodiment.

FIG. 6 is a diagram illustrating an example of a shade guide used for determining the color of a dental prosthesis, according to an embodiment.

FIG. 7 is a flowchart of an operation of changing the color of an attribute control area of a three-dimensional intraoral model to a color of an object, according to an embodiment.

FIG. 8 is a diagram illustrating an example of a screen including a first three-dimensional intraoral model displayed on the display of the data processing device, according to an embodiment.

FIG. 9 is a diagram illustrating an example of arranging a shade guide in front of teeth of a patient and scanning the teeth, according to an embodiment.

FIG. 10 is a diagram illustrating examples of a first three-dimensional intraoral model and a second three-dimensional intraoral model, according to an embodiment.

FIG. 11 is a reference diagram for describing a method of changing the color of an attribute control area of a first three-dimensional intraoral model by using a second three-dimensional intraoral model, according to an embodiment.

FIG. 12 is a diagram illustrating an example of a first three-dimensional intraoral model in which the color of an attribute control area is changed, according to an embodiment.

FIG. 13 is a reference diagram for describing a cord packing operation according to an embodiment.

FIG. 14 is a flowchart of an example of an operating method using an attribute control area in a cord packing operation, according to an embodiment.

FIG. 15 is a diagram illustrating an example in which the color an attribute control area is changed to a target color, according to an embodiment.

FIG. 16 is a diagram illustrating an example of a three-dimensional intraoral model obtained by scanning an oral cavity with teeth having holes thereon, according to an embodiment.

FIG. 17 is an example of an operation flowchart of a method of using an attribute control area for an impression scan, according to an embodiment.

FIG. 18 is a diagram illustrating an example of changing the color of a tooth by setting an attribute control area for an impression scan, according to an embodiment.

### MODE OF DISCLOSURE

In the present specification, the principle of the disclosure is described and embodiments are provided in such a manner that the scope of the present disclosure becomes apparent and the present disclosure may be carried out by those of skill in the art to which the present disclosure pertains. The disclosed embodiments may be implemented in various forms.

Like reference numerals denote like elements throughout the present specification. The present specification does not describe all elements of embodiments, and general content in the art to which the present disclosure pertains or identical content between the embodiments will be omitted. The terms ‘part’ and ‘portion’ as used herein may be embodied as software or hardware, and a plurality of ‘parts’ may be embodied as a single unit or element, while a single ‘part’ may include a plurality of elements, according to embodiments. Hereinafter, an operating principle and embodiments of the present disclosure will be described with reference to the accompanying drawings.

In the present specification, an image (hereinafter, referred to as an ‘intraoral image’) may include an image representing at least one tooth or an oral cavity including at least one tooth.

In addition, in the present specification, an image may be a two-dimensional image of an object or a three-dimensional model or three-dimensional image representing the object in three dimensions. In addition, in the present specification, an image may refer to data required to represent an object in two dimensions or three dimensions, for example, raw data obtained by at least one image sensor. In detail, the raw data is data obtained to generate an intraoral image, and may be data (e.g., two-dimensional data) obtained by at least one image sensor included in an intraoral scanner when a patient's oral cavity that is an object is scanned by using the intraoral scanner.

In the present specification, an ‘object’ may include a tooth, gingiva, at least a partial area of an oral cavity, and/or an artificial structure insertable into an oral cavity (e.g., an orthodontic device, an implant, an artificial tooth, an orthodontic auxiliary tool inserted into an oral cavity, etc.). Here, the orthodontic device may include at least one of a bracket, an attachment, an orthodontic screw, a lingual orthodontic device, and a removable orthodontic retainer.

Hereinafter, embodiments will be described in detail with reference to the drawings.

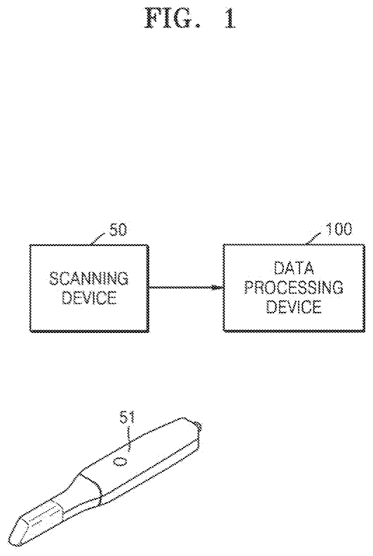

FIG. 1 is a diagram for describing a digital intraoral model processing system according to the disclosed embodiment.

Referring to FIG. 1, the digital intraoral model processing system may include a scanning device 50 and a data processing device 100.

The scanning device 50 is a device for scanning teeth, and may be an intraoral scanner for scanning a patient's teeth by being inserted into the patient's oral cavity, or a model scanner for installing a dental model and scanning while moving around the installed dental model.

The scanning device 50 may use an optical triangulation technique or a confocal technique for measuring three-dimensional information of an object. The optical triangulation technique is for obtaining three-dimensional information of an object through triangulation using a triangle formed by a light source, an object irradiated with light emitted from the light source, and an image sensor to which light reflected from the object is input, and the confocal technique is for obtaining three-dimensional information of an object based on the position of a point identified through maximum intensity of reflected light according to the refractive index of a lens that passes light emitted toward the object.

For example, an intraoral scanner 51 may be a device for obtaining an image of an oral cavity including at least one tooth, by being inserted into the oral cavity and scanning teeth in a contactless manner. In addition, the intraoral scanner 51 may have a shape capable of being drawn in and out of an oral cavity, and scans the oral cavity of a patient by using at least one image sensor (e.g., an optical camera, etc.). The intraoral scanner 51 may obtain, as raw data, surface information about an object, in order to image the surface of at least one of a tooth, gingiva, and an artificial structure (e.g., an orthodontic device including a bracket and a wire, an implant, an artificial tooth, an orthodontic auxiliary tool inserted into the oral cavity, etc.).

Image data obtained by the scanning device 50 may be transmitted to the data processing device 100 connected thereto through a wired or wireless communication network.

The data processing device 100 may be any device capable of connecting to the scanning device 50 through a wired or wireless communication network, receiving, from the scanning device 50, a two-dimensional image obtained by scanning an oral cavity, and generating, processing, displaying, and/or transmitting an intraoral image based on the received two-dimensional image.

Based on two-dimensional image data received from the scanning device 50, the data processing device 100 may generate at least one of information generated by processing the two-dimensional image data and an intraoral image generated by processing the two-dimensional image data, and display the generated information and intraoral image through a display.

The data processing device 100 may be a computing device such as a smart phone, a laptop computer, a desktop computer, a personal digital assistant (PDA), or a tablet personal computer (PC), but is not limited thereto.

In addition, the data processing device 100 may be in the form of a server (or a server device) or the like for processing an intraoral image.

In addition, the scanning device 50 may transmit, to the data processing device 100, raw data obtained through scanning, as it is. In this case, the data processing device 100 may generate a three-dimensional intraoral image representing the oral cavity in three dimensions, based on the received raw data. In addition, the ‘three-dimensional intraoral image’ may be generated by modeling the internal structure of the oral cavity in three dimensions based on the received raw data, and thus may be referred to as a ‘three-dimensional intraoral model’, a ‘digital intraoral model’, or a ‘three-dimensional intraoral image’. Hereinafter, a model or an image representing an oral cavity in two dimensions or three dimensions will be collectively referred to as an ‘intraoral image’.

In addition, the data processing device 100 may analyze, process, display, and/or transmit the generated intraoral image to an external device.

As another example, the scanning device 50 may obtain raw data through scanning, process the obtained raw data to generate an image corresponding to the oral cavity that is an object, and transmit the image to the data processing device 100. In this case, the data processing device 100 may analyze, process, display, and/or transmit the received image.

In the disclosed embodiment, the data processing device 100 is an electronic device capable of generating and displaying an intraoral image representing an oral cavity including one or more teeth in three dimensions, and will be described in detail below.

According to an embodiment, when receiving raw data obtained by scanning an oral cavity from the scanning device 50, the data processing device 100 may process the received raw data to generate a three-dimensional intraoral model.

According to an embodiment, the data processing device 100 may set at least a partial area in the generated three-dimensional intraoral model, and control an attribute of the set area.

According to an embodiment, the data processing device 100 may set at least a partial area in the three-dimensional intraoral model, an attribute of which is to be controlled. According to an embodiment, the data processing device 100 may set, based on a user input, a partial area of which an attribute is to be controlled. According to an embodiment, the data processing device 100 may automatically set a partial area of which an attribute is to be controlled. Such a set area of which an attribute is to be controlled is for changing or controlling the attribute of the area in the three-dimensional intraoral model, rather than using the attribute of the area as it is, and may be referred to as an “attribute control area”. Hereinafter, such an area will be referred to as an attribute control area.

According to an embodiment, the data processing device 100 may change or control an attribute of at least a partial area of the three-dimensional intraoral model that is set for controlling the attribute, i.e., the attribute control area. The attributes of an area of the three-dimensional intraoral model may include a position and a color of data included in the area. According to an embodiment, the three-dimensional intraoral model may have a mesh structure consisting of a plurality of vertices, and an area of the three-dimensional intraoral model may include a plurality of vertices included in the area. In addition, each vertex may have position information and color information. Thus, in detail, the data processing device 100 may change or control attributes of vertices included in the attribute control area, i.e., positions or colors.

According to an embodiment, in order to change or control the attribute of the attribute control area set in the three-dimensional intraoral model, the data processing device 100 may obtain a second three-dimensional intraoral model and control the attribute of the attribute control area based on an attribute of an area in the obtained second three-dimensional intraoral model corresponding to the position of the attribute control area.

According to an embodiment, the data processing device 100 may change a color of a partial dental area of the three-dimensional intraoral model to a color of a dental area of the second three-dimensional intraoral model corresponding to the partial dental area. The data processing device 100 may set a partial dental area of the three-dimensional intraoral model as an attribute control area. The data processing device 100 may obtain a second three-dimensional intraoral model obtained by overlaying an object on a tooth or a dental model part set as an attribute control area, and performing a scan. The data processing device 100 may identify an area in the second three-dimensional intraoral model corresponding to the attribute control area, extract color information of the identified area, and change a color of a dental area set as the attribute control area of the three-dimensional intraoral model, to a color extracted from the second three-dimensional intraoral model. In this way, the data processing device 100 may change the color of the dental area set as the attribute control area of the three-dimensional intraoral model, to the color of the object. As such, by changing the color of the dental area set as the attribute control area in the three-dimensional intraoral model to the color of the object, a user may more reliably compare the color of an adjacent dental area adjacent to the dental area with the color of the object. For example, the object may include a shade guide used to determine the color of a dental prosthesis.

According to an embodiment, the data processing device 100 may set a partial dental area of the three-dimensional intraoral model as an attribute control area. The data processing device 100 may obtain a second three-dimensional intraoral model by scanning the dental area set as the attribute control area after foreign substances are removed from a tooth corresponding to the dental area. The data processing device 100 may identify an area in the second three-dimensional intraoral model corresponding to the attribute control area, extract color information of the identified area, and change a color of a dental area set as the attribute control area of the three-dimensional intraoral model, to a color extracted from the second three-dimensional intraoral model. In this way, the data processing device 100 may change the color of the dental area set as the attribute control area of the three-dimensional intraoral model, to the color of a tooth from which foreign substances are removed. As such, by changing the color of the dental area set as the attribute control area in the three-dimensional intraoral model to the color of a tooth from which foreign substances are removed, the user may obtain a three-dimensional intraoral model from which the foreign substances are removed, through a simple operation. Here, for example, the dental area set as the attribute control area may include a margin area of the three-dimensional intraoral model. In this way, the user may conveniently changing an attribute of an area including foreign substances such as blood or saliva due to a cord packing operation, to an attribute from which the foreign substances are removed.

According to an embodiment, the data processing device 100 may obtain a three-dimensional intraoral model by scanning an impression, and set a partial dental area of the obtained three-dimensional intraoral model as an attribute control area. The data processing device 100 may obtain a second three-dimensional intraoral model by scanning teeth corresponding to the impression. The data processing device 100 may identify an area in the second three-dimensional intraoral model corresponding to the attribute control area, extract color information of the identified area, and change a color of the dental area set as the attribute control area of the three-dimensional intraoral model, to a color extracted from the second three-dimensional intraoral model. In this way, the data processing device 100 may change the color of the dental area set as the attribute control area of the three-dimensional intraoral model corresponding to the impression, to the color of a tooth of the second three-dimensional intraoral model corresponding to an actual tooth. As such, by changing the color of the dental area set as the attribute control area in the three-dimensional intraoral model corresponding to the impression to the color of the tooth of the second three-dimensional intraoral model obtained by scanning the teeth, the user may change the color of the teeth of the three-dimensional intraoral model corresponding to the impression to the color of the actual teeth.

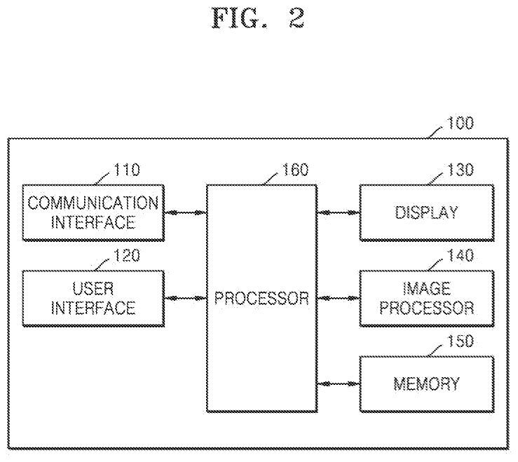

FIG. 2 is a block diagram illustrating the data processing device 100 according to an embodiment.

Referring to FIG. 2, the data processing device 100 may include a communication interface 110, a user interface 120, a display 130, an image processor 140, a memory 150, and a processor 160.

The communication interface 110 may perform communication with at least one external electronic device through a wired or wireless communication network. In detail, the communication interface 110 may communicate with the scanning device under control of the processor 160. The communication interface 110 may perform communication with an external electronic device or server connected thereto through a wired/wireless communication network, under control of the processor.

The communication interface 110 may communicate with an external electronic device (e.g., an intraoral scanner, a server, or an external medical device) through a wired or wireless communication network. In detail, the communication interface may include at least one short-range communication module configured to perform communication according to a communication standard such as Bluetooth, Wi-Fi, Bluetooth Low Energy (BLE), near-field communication (NFC)/radio-frequency identification (RFID), Wi-Fi Direct, ultra-wideband (UWB), or Zigbee.

In addition, the communication interface 110 may further include a long-range communication module configured to communicate with a server for supporting long-range communication according to a long-range communication standard. In detail, the communication interface 110 may include a long-range communication module configured to perform communication through a network for Internet communication. In addition, the communication interface may include a communication module configured to perform communication through a communication network conforming to a mobile communication standard, such as 3^{rd} Generation (3G), 4^{th} Generation (4G), and/or 5^{th} Generation (5G).

In addition, the communication interface 110 may include at least one port for connecting to an external electronic device (e.g., an intraoral scanner, etc.) through a cable in order to communicate with the external electronic device. As such, the communication interface 110 may communicate with an external electronic device connected thereto in a wired manner through the at least one port.

The user interface 120 may receive a user input for controlling the data processing device. The user interface 120 may include, but is not limited to, a touch panel for detecting a touch of the user, a button for receiving a push manipulation by the user, and a user input device including a mouse or a keyboard for designating or selecting a point on a user interface screen.

In addition, the user interface 120 may include a speech recognition device for speech recognition. For example, the speech recognition device may be a microphone, and may receive a voice command or a voice request of a user. Accordingly, the processor may control an operation corresponding to the voice command or voice request to be performed.

The display 130 displays a screen. In detail, the display 130 may display a certain screen under control of the processor 160. In detail, the display 130 may display a user interface screen including an intraoral image generated based on data obtained by the scanning device 50 scanning the oral cavity of the user. Alternatively, the display 130 may display a user interface screen including information related to dental treatment for the patient.

The image processor 140 may perform operations for generating and/or processing an image. In detail, the image processor 140 may receive raw data obtained by the scanning device 50 and generate a three-dimensional intraoral model based on the received raw data. The image processor 140 may be provided separately from the processor 160 as illustrated in FIG. 2, or may be included in the processor 160.

The memory 150 may store at least one instruction. In addition, the memory 150 may store at least one instruction executable by the processor. Also, the memory may store at least one program executable by the processor 160. Also, the memory 150 may store data (e.g., raw data obtained through intraoral scanning) received from an intraoral scanner. Alternatively, the memory may store an intraoral image representing an oral cavity in three dimensions.

The processor 160 executes the at least one instruction stored in the memory 150 to perform control such that an intended operation is performed. Here, the at least one instruction may be stored in an internal memory included in the processor 160, or in the memory 150 included in the data processing device separately from the processor.

In detail, the processor 160 may execute the at least one instruction to control at least one component included in the data processing device such that an intended operation is performed. Therefore, although an example in which the processor performs certain operations is described, it may mean that the processor controls at least one component included in the data processing device to perform the operations.

According to an embodiment, the processor 160 execute one or more instructions stored in the memory 150 to display a first three-dimensional intraoral model on a display, receive an input for setting at least a part of the displayed first three-dimensional intraoral model as an attribute control area, obtain a second three-dimensional intraoral model, change a color of the at least a part of the first three-dimensional intraoral model set as the attribute control area, to a target color representing a color of an area of the obtained second three-dimensional intraoral model corresponding to the attribute control area, and display, on the display, the first three-dimensional intraoral model in which the color of the attribute control area is changed to the target color.

According to an embodiment, the processor 160 execute the one or more instructions stored in the memory 150 to identify the area of the obtained second three-dimensional intraoral model corresponding to the attribute control area, and obtain the target color by extracting color information from the identified area.

According to an embodiment, the processor 160 execute the one or more instructions stored in the memory 150 to obtain the second three-dimensional intraoral model based on data obtained by scanning a tooth in a state in which an object is arranged to be overlaid on a front surface of the tooth. According to an embodiment, the object may be arranged at a tooth position corresponding to a position set as the attribute control area in the three-dimensional intraoral model.

According to an embodiment, the processor 160 execute the one or more instructions stored in the memory 150 to identify a position set as the attribute control area in the first three-dimensional intraoral model, and obtain the target color by extracting color information from an area of the second three-dimensional intraoral model corresponding to the identified position.

According to an embodiment, the processor 160 execute the one or more instructions stored in the memory 150 to receive an input for setting the attribute control area in the first three-dimensional intraoral model obtained by scanning a tooth on which cord packing has been performed.

According to an embodiment, the processor 160 execute the one or more instructions stored in the memory 150 to obtain the second three-dimensional intraoral model generated by scanning a tooth from which foreign substances are removed, obtain the target color by extracting color information from the area of the obtained second three-dimensional intraoral model corresponding to the attribute control area, and change the color of the at least a part of the first three-dimensional intraoral model set as the attribute control area to the obtained target color.

According to an embodiment, the first three-dimensional intraoral model may include a three-dimensional intraoral model obtained by scanning a dental impression.

According to an embodiment, the processor 160 execute the one or more instructions stored in the memory 150 to obtain the second three-dimensional intraoral model by scanning a tooth, obtain the target color by extracting color information from the area of the obtained second three-dimensional intraoral model corresponding to the attribute control area, and change the color of the at least a part of the first three-dimensional intraoral model set as the attribute control area to the obtained target color.

According to an embodiment, the processor 160 may be implemented to include at least one internal processor and a memory device (e.g., random-access memory (RAM), read-only memory (ROM), etc.) for storing at least one of programs, instructions, signals, and data to be processed or used by the internal processor.

In addition, the processor 160 may include a graphics processing unit for performing graphics processing on a video. In addition, the processor may be implemented as a system on chip (SoC) in which a core and a graphics processing unit (GPU) are integrated. In addition, the processor may include a single processor core (single-core) or a plurality of processor cores (multi-core). For example, the processor may be dual-core, triple-core, quad-core, hexa-core, octa-core, deca-core, dodeca-core, hexadecimal-core, or the like.

In the disclosed embodiment, the processor 160 may generate a three-dimensional intraoral model based on a two-dimensional image received from the scanning device 50.

In detail, under control of the processor 160, the communication interface 110 may receive data obtained by the scanning device 50, for example, raw data obtained through intraoral scanning. In addition, the processor 160 may generate a three-dimensional intraoral image representing an oral cavity in three dimensions, based on the raw data received by the communication interface. For example, in order to reconstruct a three-dimensional image by performing an optical triangulation technique, the intraoral scanner may include at least one camera, and according to a specific embodiment, may include an L camera corresponding to a left field of view and an R camera corresponding to a right field of view. In addition, the intraoral scanner may obtain L image data corresponding to the left field of view and R image data corresponding to the right field of view, from the L camera and the R camera, respectively. Subsequently, the intraoral scanner (not shown) may transmit raw data including the L image data and the R image data, to the communication interface of the data processing device 100.

Then, the communication interface 110 may transmit the received raw data to the processor, and the processor may generate a three-dimensional intraoral model representing the oral cavity in three dimensions, based on the received raw data.

In addition, the processor 160 may control the communication interface to directly receive an intraoral image representing an oral cavity in three dimensions, from an external server, medical device, or the like. In this case, the processor may obtain a three-dimensional intraoral model without generating the three-dimensional intraoral model based on raw data.

According to the disclosed embodiment, that the processor 160 perform operations such as ‘extraction’, ‘obtaining’, and ‘generation’ may mean that the processor 160 executes at least one instruction to directly perform the operations, as well as that the processor 160 controls other components such that the operations are performed.

In order to implement the embodiments disclosed herein, the data processing device 100 may include only some of the components illustrated in FIG. 2 or may include more components than the components illustrated in FIG. 2.

In addition, the data processing device 100 may store and execute dedicated software linked to the intraoral scanner. Here, the dedicated software may be referred to as a dedicated program, a dedicated tool, or a dedicated application. In a case in which the data processing device 100 operates in conjunction with the scanning device 50, the dedicated software stored in the data processing device 100 may be connected to the scanning device 50 to receive data obtained through intraoral scanning in real time.

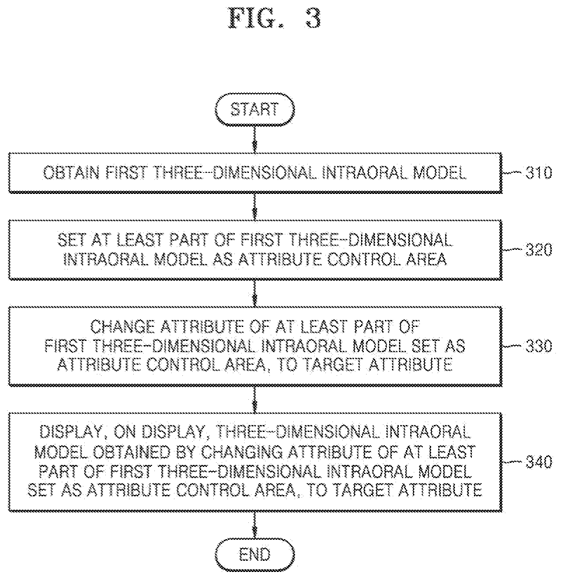

FIG. 3 is a flowchart illustrating a method, performed by a data processing device, of processing a three-dimensional intraoral model, according to the disclosed embodiment. The method of processing a three-dimensional intraoral model illustrated in FIG. 3 may be performed by the data processing device 100. Thus, the flowchart of the method of processing a three-dimensional intraoral model illustrated in FIG. 3 may be a flowchart of operations performed by the data processing device 100.

Referring to FIG. 3, in operation 310, the data processing device 100 may obtain a first three-dimensional intraoral model.

The data processing device 100 may receive, from the scanning device 50, raw data obtained by scanning the oral cavity of a patient or by scanning a dental model, and process the received raw data to obtain a first three-dimensional intraoral model including a dental area and a gingival area.

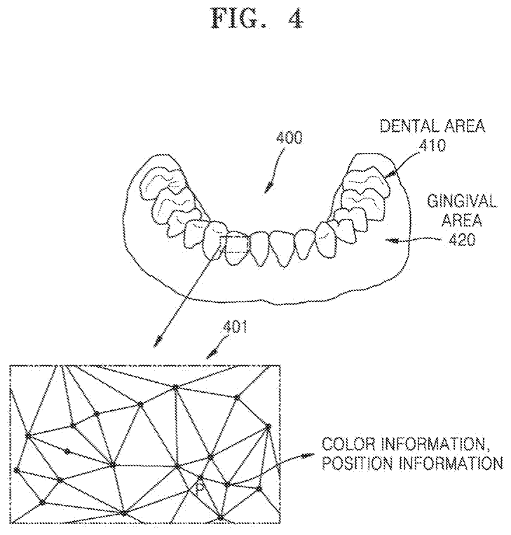

FIG. 4 is a diagram illustrating an example of a first three-dimensional intraoral model obtained by the data processing device 100, according to an embodiment.

For example, when two-dimensional data is obtained by using an intraoral scanner, the data processing device 100 may calculate coordinates of a plurality of illuminated surface points by using a triangulation method. As the amount of scan data increases by scanning the surface of an object by using the intraoral scanner while moving the intraoral scanner, coordinates of the surface points may be accumulated. As a result of the obtaining of images, a point cloud of vertices may be identified to represent the extent of the surface. Points in the point cloud may represent actual measured points on the three-dimensional surface of the object. The surface structure may be approximated by forming a polygon mesh in which adjacent vertices of the point cloud are connected to each other by line segments. The polygon mesh may be variously determined, such as a triangle, quadrangle, or pentagon mesh. Relationships between adjacent polygons of such a mesh model may be used to extract features of a tooth boundary, such as curvature, minimum curvature, edge, spatial relation, etc.

Referring to FIG. 4, a first three-dimensional intraoral model 400 may include a dental area 410 and a gingival area 420. A partial area 401 of the first three-dimensional intraoral model 400 may be configured as a triangle mesh formed by connecting a plurality of vertices constituting a point cloud to adjacent vertices with lines. Each vertex may include position information and color information as its attributes. The position information that each vertex has as an attribute may be composed of X, Y, and Z coordinates on a three-dimensional coordinate system. The color information that each vertex has as an attribute may have red-green-blue (RGB) values representing a color obtained by a camera or an image sensor included in the scanning device. As such, the shape, outline, and color of the first three-dimensional intraoral model 400 may be represented by the attributes of each vertex, i.e., position information and color information.

For example, a mesh may be formed by vertices, and color information of the mesh may be generated or represented by a value of each vertex. For example, when a mesh is formed by three vertices, color information of the mesh may be generated by using values of one or more vertices among the three vertices constituting the mesh. For example, color information of the mesh may be generated by using an average value of color values of the three vertices.

Referring back to FIG. 3, in operation 320, the data processing device 100 may set at least a part of the first three-dimensional intraoral model as an attribute control area.

According to an embodiment, the data processing device 100 may display the obtained first three-dimensional intraoral model on a display, and receive a user input for selecting an area of the displayed first three-dimensional intraoral model to be set as an attribute control area. For example, the data processing device 100 may provide a graphical user interface for selecting an attribute control area, and receive a user input for selecting an attribute control area through the provided graphical user interface.

According to an embodiment, the data processing device 100 may automatically set an attribute control area in the obtained first three-dimensional intraoral model by itself. According to an embodiment, the data processing device 100 may evaluate the reliability of data constituting the first three-dimensional intraoral model, and set, as an attribute control area, an area in which the reliability of data is evaluated to be greater than a threshold value.

When an object is scanned by the scanning device, more data is accumulated as an area is repeatedly scanned, and thus, the reliability of data of the area may increase. The data processing device 100 may obtain a data density representing the degree of accumulation of data of a unit area obtained by scanning, and may evaluate the reliability of the unit area based on the data density. For example, the data processing device 100 may determine the threshold value for evaluating the reliability of the data constituting the first three-dimensional intraoral model, and compare the data density of a preset unit area of the data constituting the first three-dimensional intraoral model with the predetermined threshold value. The data processing device 100 may set, as an attribute control area, an area in which the reliability of a unit area is determined, based on a result of the comparing, to be greater than the threshold value.

According to an embodiment, the data processing device 100 may set a partial area of the first three-dimensional intraoral model as an attribute control area, or may set the entire area of the first three-dimensional intraoral model as an attribute control area.

In operation 330, the data processing device 100 may change an attribute of the at least a part of the first three-dimensional intraoral model set as the attribute control area, to a target attribute.

In detail, the data processing device 100 may change attributes of vertices included in the area set as the attribute control area in the first three-dimensional intraoral model to target attributes.

According to an embodiment, the data processing device 100 may change the color of the at least a part of the first three-dimensional intraoral model set as the attribute control area, to a target color.

According to an embodiment, the data processing device 100 may change both the color and position information of the at least a part of the first three-dimensional intraoral model set as the attribute control area, to a target color and target position information, respectively.

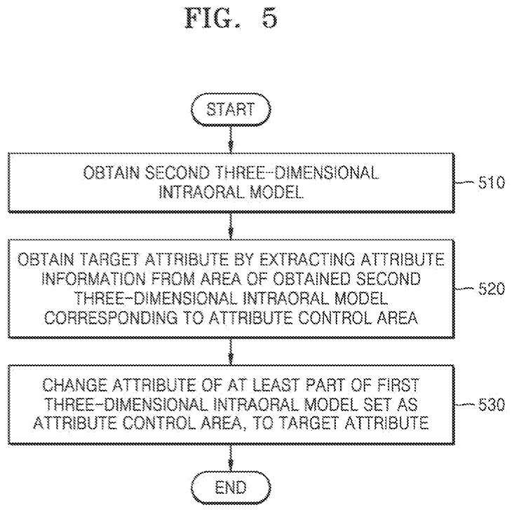

A method of obtaining a target color to which the color of an attribute control area is to be changed will be described in detail below with reference to FIG. 5.

In operation 340, the data processing device 100 may display, on the display, a three-dimensional intraoral model obtained by changing an attribute of the at least a part of the first three-dimensional intraoral model set as the attribute control area, to the target attribute.

As such, the data processing device 100 changes an attribute of the attribute control area set in the first three-dimensional intraoral model and displays the resulting three-dimensional intraoral model on the display, such that the user who wants to change a partial area of the previously obtained first three-dimensional intraoral model obtains a desired three-dimensional intraoral model more conveniently without going through the scanning process again from the beginning.

FIG. 5 is a flowchart of an example of a method of obtaining a target color for controlling an attribute of an attribute control area set in a part of a first three-dimensional intraoral model, according to an embodiment.

Referring to FIG. 5, in operation 510, the data processing device 100 may obtain a second three-dimensional intraoral model. The second three-dimensional intraoral model may be about the entire area or a partial area of an object that is a target of a first three-dimensional intraoral model.

According to an embodiment, in a case in which the target of the first three-dimensional intraoral model is the oral cavity or a dental model of a patient, the data processing device 100 may obtain the second three-dimensional intraoral model by overlaying an object on a partial area of the oral cavity or dental model of the patient, i.e., an area corresponding to an attribute control area, and scanning the oral cavity or dental model of the patient on which the object is overlaid. As such, when scanning an area on which an object is overlaid to obtain the second three-dimensional intraoral model, it is sufficient to scan only the area on which the object is overlaid and an adjacent area, without having to scan the entire oral cavity or dental model. Of course, it is also possible to scan the entire oral cavity or dental model.

According to an embodiment, in a case in which the target of the first three-dimensional intraoral model is the oral cavity or a dental model of a patient, the data processing device 100 may obtain the second three-dimensional intraoral model by scanning the oral cavity or dental model of the patient in a state in which foreign substances are removed from a partial area of the oral cavity or dental model of the patient, i.e., an area corresponding to an attribute control area. For example, in a case in which teeth of the patient are stained with blood or saliva when generating a first three-dimensional intraoral model, a second three-dimensional intraoral model may be obtained by setting, as an attribute control area, an area of the teeth stained with the blood or saliva, and scanning only an area corresponding to the attribute control area after the blood or saliva is removed from the teeth of the patient.

According to an embodiment, in a case in which the target of the first three-dimensional intraoral model is an impression, the data processing device 100 may obtain a second three-dimensional intraoral model by scanning the oral cavity of the patient corresponding to the impression.

In operation 520, the data processing device 100 may obtain a target attribute by extracting attribute information from an area of the obtained second three-dimensional intraoral model corresponding to the attribute control area. The attribute information of the corresponding area may include color information and/or position information.

In detail, the data processing device 100 may identify the position information of the area of the first three-dimensional intraoral model set as the attribute control area. In addition, the data processing device 100 may identify an area of the second three-dimensional intraoral model corresponding to the identified position information, and extract attribute information from the identified area.

In operation 530, the data processing device 100 may change an attribute of the at least a part of the first three-dimensional intraoral model set as the attribute control area, to the obtained target attribute.

According to an embodiment, the data processing device 100 may change the color of the at least a part of the first three-dimensional intraoral model set as the attribute control area, to an obtained target color.

The data processing device 100 may further accumulate data included in other areas of the second three-dimensional intraoral model than the attribute control area of the first three-dimensional intraoral model, i.e., other areas of the second three-dimensional intraoral model than the area corresponding to the attribute control area, in areas of the first three-dimensional intraoral model corresponding to the other areas of the second three-dimensional intraoral model, so as to reflect the data in the first three-dimensional intraoral model. For example, because each vertex includes position information and color information, only the color information of the corresponding area of the second three-dimensional intraoral model is reflected in the attribute control area of the first three-dimensional intraoral model, and both the position information and the color information of the other areas of the second three-dimensional intraoral model may be reflected in the other areas of the first three-dimensional intraoral model than the attribute control area.

Hereinafter, examples that may be applied to dental treatment or treatment planning using an attribute control area as described above will be described.

According to an embodiment, for determining the color of a dental prosthesis, an attribute control area may be used as described above.

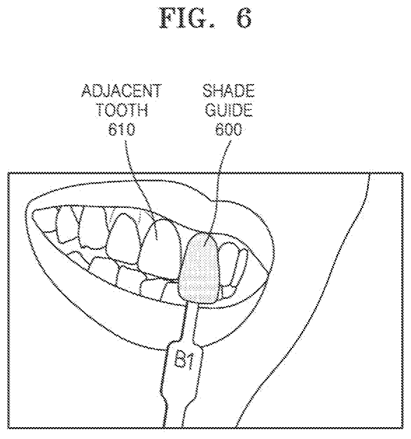

FIG. 6 is a diagram illustrating an example of a shade guide used for determining the color of a dental prosthesis, according to an embodiment.

In producing a dental prosthesis for dental treatment, it is important not only to determine the shape of the dental prosthesis but also to determine the color of the dental prosthesis. In particular, in producing a dental prosthesis for an anterior tooth, selecting the color of the dental prosthesis to be as similar as possible to the original tooth of the patient is significantly important because it may greatly affect the patient's satisfaction with the dental prosthesis.

Referring to FIG. 6, when selecting the color of a dental prosthesis, a dentist usually holds up a shade guide 600 with various colors to a position in the oral cavity of a patient where the dental prosthesis is to be placed, and select a shade with the most similar color to a current tooth of the patient, for example, an adjacent tooth 610. The shade guide refers to a model tooth expressing the degree of lightness and darkness of a tooth in stages. The color of tooth is determined by various factors such as whiteness, brightness, lightness, or transparency, accordingly, the shade guide usually consists of more than ten stages, and thus, it is not easy to determine a shade suitable for teeth of a patient. In addition, when holding up a shade guide to the teeth of the patient for determining the shade, it may be difficult for the patient and the dentist to accurately select the color with the naked eye.

As described above, in determining the color of a dental prosthesis, it may be useful to allow the patient and the dentist to determine the color of the dental prosthesis by setting an attribute control area in a three-dimensional virtual model and changing the color of the set attribute control area to the color of a shade guide so as to compare the color of the tooth of the patient with the shade guide on one screen, rather than simply holding up the shade guide to the tooth of the patient and determining the color on the dental prosthesis.

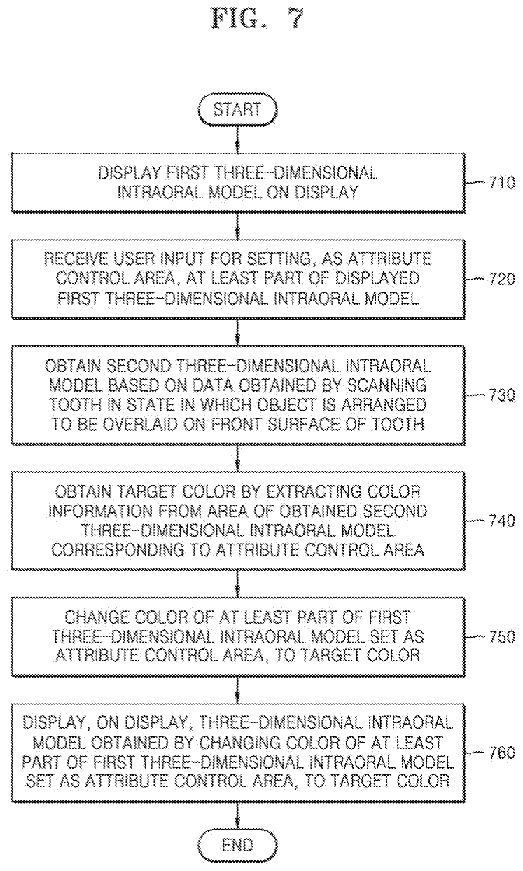

FIG. 7 is a flowchart of an operation of changing the color of an attribute control area of a three-dimensional intraoral model to a color of an object, according to an embodiment. Among the operations illustrated in FIG. 7, those similar to the operations illustrated in FIGS. 3 and 5 will be briefly described.

Referring to FIG. 7, in operation 710, the data processing device 100 may display a first three-dimensional intraoral model on the display. For example, the data processing device 100 may generate the first three-dimensional intraoral model based on data obtained by scanning the oral cavity of a patient, and display the generated first three-dimensional intraoral model on the display.

In operation 720, the data processing device 100 may receive a user input for setting, as an attribute control area, at least a part of the first three-dimensional intraoral model displayed on the display.

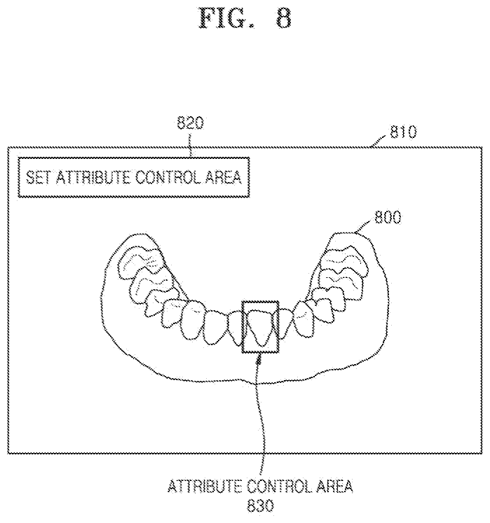

FIG. 8 is a diagram illustrating an example of a screen including a first three-dimensional intraoral model displayed on the display of the data processing device 100, according to an embodiment.

Referring to FIG. 8, the data processing device 100 may output a screen 810 displaying a first three-dimensional intraoral model 800, to the display. The screen 810 may include a menu 820 for setting at least a part of the first three-dimensional intraoral model 800 as an attribute control area. When the user selects the menu 820, the user may designate an attribute control area 830 through various input units, such as a mouse, a keyboard, or a touch input interface. The user may set at least a partial area of the first three-dimensional intraoral model 800 as an attribute control area. For example, as illustrated in FIG. 8, the user may set an area corresponding to one of anterior teeth, as an attribute control area. Of course, the user may set the entire area of the first three-dimensional intraoral model as an attribute control area.

According to an embodiment, the user may set an attribute control area at a tooth position in the first three-dimensional intraoral model 800 where a dental prosthesis is to be placed.

In operation 730, the data processing device 100 may obtain a second three-dimensional intraoral model based on data obtained by scanning a tooth in a state in which an object is arranged to be overlaid on the front surface of the tooth.

The object arranged in front of the tooth may have various shapes or colors. For selecting the color of the dental prosthesis, for example, the object may include a shade guide.

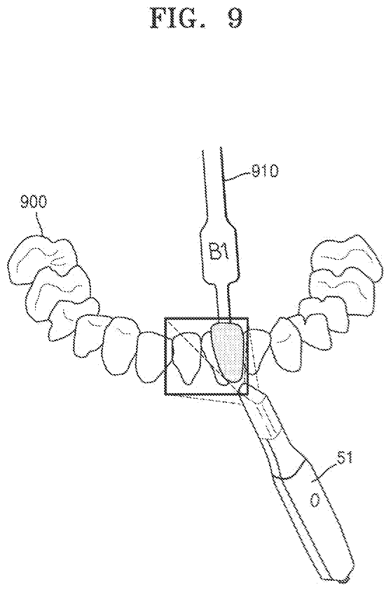

FIG. 9 is a diagram illustrating an example of arranging a shade guide in front of teeth of a patient and scanning the teeth, according to an embodiment.

Referring to FIG. 9, the intraoral scanner 51 may obtain image data in which a shade guide is overlaid, by scanning the teeth of the patient in a state in which a shade guide 910 is overlaid on a partial area of teeth 900 of the patient, i.e., a position where a dental prosthesis is to be placed or a position set as an attribute control area. Here, the intraoral scanner 51 may scan all teeth overlaid with the shade guide 910, or may scan only a part of an adjacent area including the shade guide 910. In order to identify, in a subsequent operation, the area corresponding to the attribute control area, in the two-dimensional intraoral model obtained by scanning the teeth overlaid with the shade guide, it is preferable to scan not only the area corresponding to the attribute control area but also at least a part of an adjacent area. The example of FIG. 9 in which the shade guide 910 is overlaid on the tooth is an example, and the present disclosure is not limited thereto. For example, the shade guide 910 may be produced to be attachable to a tooth of a patient, such that the attachable shade guide may be attached to a tooth of a patient, and then an adjacent area including the tooth to which the shade guide 910 is attached may be scanned.

The data processing device 100 may receive, from the intraoral scanner 51, image data obtained by scanning the teeth in a state in which the shade guide 910 is arranged to be overlaid, and obtain a second three-dimensional intraoral model based on the received image data.

In operation 740, the data processing device 100 may obtain a target color by extracting color information from an area of the obtained second three-dimensional intraoral model corresponding to the attribute control area.

In detail, the data processing device 100 may identify the position of the area of the first three-dimensional intraoral model set as the attribute control area. In addition, the data processing device 100 may identify an area of the second three-dimensional intraoral model corresponding to the position of the area of the first three-dimensional intraoral model set as the attribute control area. The data processing device may obtain a target color by extracting color information of vertices included in the corresponding area of the second three-dimensional intraoral model.

In operation 750, the data processing device 100 may change the color of the at least a part of the first three-dimensional intraoral model set as the attribute control area, to the target color. In detail, the data processing device 100 may change the color of vertices of the at least a part of the first three-dimensional intraoral model set as the attribute control area, to the obtained target color.

In operation 760, the data processing device 100 may display, on the display, a three-dimensional intraoral model obtained by changing the color of the at least a part of the first three-dimensional intraoral model set as the attribute control area, to the target color.

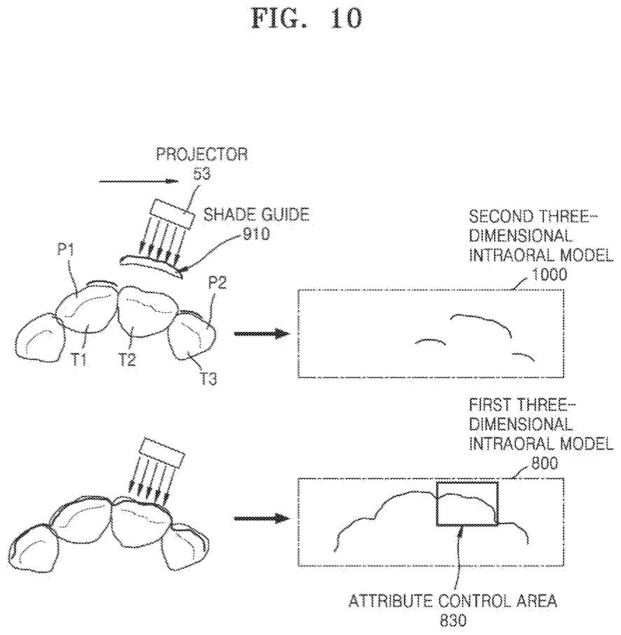

Hereinafter, detailed examples of operations 740, 750, and 760 will be described with reference to FIGS. 10 to 12.

FIG. 10 is a diagram illustrating examples of a first three-dimensional intraoral model and a second three-dimensional intraoral model, according to an embodiment.

Referring to FIG. 10, the intraoral scanner 51 projects light into the oral cavity of a patient through a projector 53, and obtains two-dimensional image data of the oral cavity through one or more cameras. Here, the intraoral scanner 51 may project light to a scan region of interest (ROI) and obtain two-dimensional image data corresponding to the scan ROI. Data obtained by the intraoral scanner 51 performing a scan while moving along the teeth of the oral cavity may be transmitted to the data processing device 100 in real time. The data processing device 100 may generate the first three-dimensional intraoral model 800 by combining one or more pieces of two-dimensional image data received in this way. In addition, a part of the first three-dimensional intraoral model may be set as the attribute control area 830. Although the first three-dimensional intraoral model 800 and the attribute control area 830 are actually in three dimensions, FIG. 10 shows a front part of the teeth in a cross-section viewed from the occlusal surface, i.e., viewed in the occlusal direction, for convenience of description.

When generating the second three-dimensional intraoral model, the intraoral scanner 51 projects light to the teeth of the patient through the projector 53 in a state in which the shade guide 910 is arranged to be overlaid in a part of the teeth of the patient, i.e., a dental area where a dental prosthesis T2 is to be placed, or an area designated as an attribute control area, and obtains two-dimensional image data of the oral cavity through one or more cameras. Data obtained by the intraoral scanner 51 performing a scan while moving along the teeth of the oral cavity may be transmitted to the data processing device 100 in real time. Here, the intraoral scanner 51 may scan all teeth of the patient, but it is sufficient to scan an area adjacent to the area where the shade guide 910 is arranged, i.e., the surrounding area, and the area where the shade guide 910 is arranged. For example, the intraoral scanner 51 may scan an area adjacent to the area where the shade guide 910 is arranged, i.e., from P1 to P2, and transmit one or more pieces of two-dimensional image data obtained as a result of the scanning, to the data processing device 100. Thus, the two-dimensional image data transmitted to the data processing device 100 may include image data for a partial area of a tooth T1 adjacent to the tooth on which the shade guide 910 is arranged, the shade guide 910, and a partial area of an adjacent tooth T3. The data processing device 100 may generate a second three-dimensional intraoral model 1000 by combining the one or more pieces of two-dimensional image data received from the intraoral scanner 51.

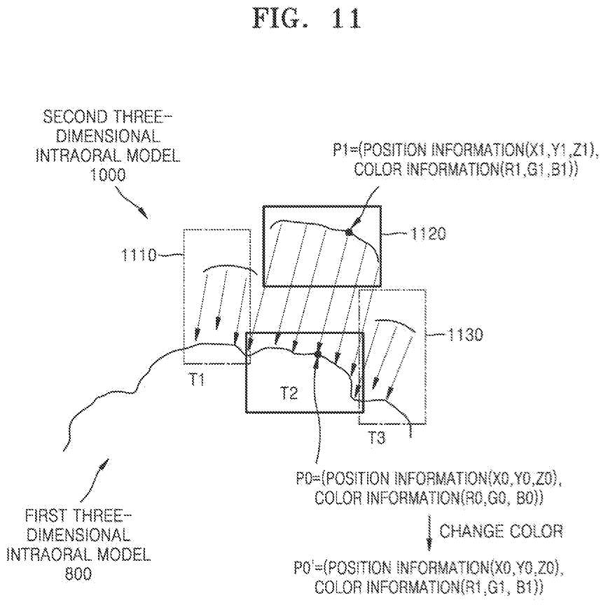

FIG. 11 is a reference diagram for describing a method of changing the color of an attribute control area of a first three-dimensional intraoral model by using a second three-dimensional intraoral model, according to an embodiment.

In order to change the color of an area of a first three-dimensional intraoral model set as an attribute control area to the color of a corresponding area of a second three-dimensional intraoral model, that is, a target color, it is necessary to identify the corresponding area in the second three-dimensional intraoral model.

For example, the data processing device 100 may identify the attribute control area by aligning the second three-dimensional intraoral model with the first three-dimensional intraoral model. In order to align the second three-dimensional intraoral model with the first three-dimensional intraoral model, the data processing device 100 may use various aligning algorithms, for example, iterative closest point (ICP). ICP is an algorithm for minimizing the difference between two point clouds, and is used to reconstruct a two-dimensional or three-dimensional surface from different pieces of scan data. The ICP algorithm fixes a point cloud, which is referred to as a reference, and transforms a point cloud, called which is referred to as a source, to best match the reference. The ICP algorithm may align a three-dimensional model by iteratively modifying transformation (a combination of translation and rotation) necessary to minimize an error metric representing the distance from the source to the reference. As the aligning algorithm, various algorithms other than ICP may be used, and for example, the Kabsch algorithm may be used.

As such, when the second three-dimensional intraoral model 1000 is aligned with the first three-dimensional intraoral model 800, an area 1110 and an area 1130 correspond to and thus may be aligned with T1 and T3, respectively. Of course, an area 1120 corresponding to T2 corresponds to the tooth T2 in the first three-dimensional intraoral model 800 and to the overlaid shade guide in the second three-dimensional intraoral model 1000, and thus may not be aligned, but both the area of T1 and the area of T3 that are adjacent to T2 may be aligned, and thus, the area corresponding to T2 may be identified.

When the area corresponding to the attribute control area of the first three-dimensional intraoral model is identified in the second three-dimensional intraoral model in this manner, the data processing device 100 may extract color information of the identified area in the second three-dimensional intraoral model, and change the color of the attribute control area of the first three-dimensional intraoral model by using the extracted color information. For example, referring to FIG. 11, assume that attribute information of a certain vertex P0 included in the attribute control area of the first three-dimensional intraoral model is (position information, color information)=((X0, Y0, Z0), (R0, G0, B0)). In addition, assume that attribute information of a vertex P1, among vertices included in the identified area in the second three-dimensional intraoral model, corresponding to P0 is (position information, color information)=((X1, Y1, Z1), (R1, G1, B1)). Then, the data processing device 100 may change the color information among attribute information of the vertex PO of the first three-dimensional intraoral model to color information of P1, which is (R1, G1, B1), thereby changing the attribute information of PO to (position information, color information)=((X0, Y0, Z0), (R1, G1, B1)).

According to an embodiment, the vertex PO of the first three-dimensional intraoral model and the vertex P1 of the second three-dimensional intraoral model that are in a relationship of color change may be two vertices in the projection direction.

According to an embodiment, the data processing device 100 may further reflect, in data of the first three-dimensional intraoral model, position information of data of the second three-dimensional intraoral model, as its attribute information, obtained from other areas than the attribute control area, i.e., the area 1110 corresponding to T1 or the area 1130 corresponding to T3. That is, the part corresponding to T1 or T3 is data corresponding to the same dental area, and thus, a more reliable first three-dimensional intraoral model may be obtained by reflecting data obtained from the second three-dimensional intraoral model in the corresponding data of the first three-dimensional intraoral model.

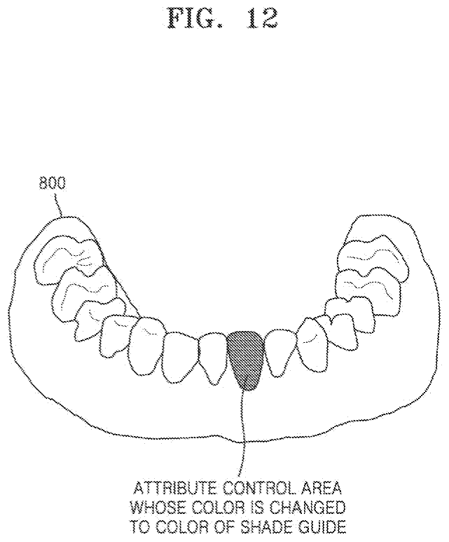

FIG. 12 is a diagram illustrating an example of a first three-dimensional intraoral model in which the color of an attribute control area is changed, according to an embodiment.

FIG. 12 illustrates that the color of the right tooth among the anterior teeth that is set as the attribute control area 830 in the first three-dimensional intraoral model 800 is changed to the color of a shade guide.

The dentist or the patient may more reliably determine whether the color of the shade guide naturally matches the teeth of the patient, while checking an image of the three-dimensional intraoral model in which the color of the part in which a dental prosthesis is to be placed, to the color of the shade guide.

According to an embodiment, such an attribute control area may be used for a dental area that is stained with foreign substances in a cord packing operation.

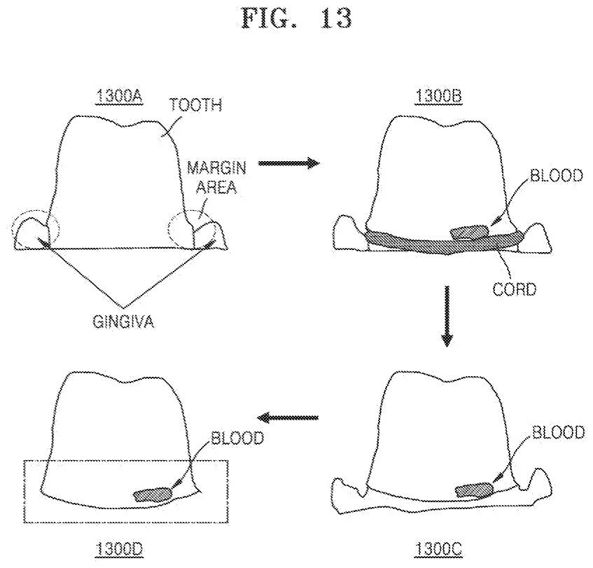

FIG. 13 is a reference diagram for describing a cord packing operation according to an embodiment.

Cord packing refers to inserting a cord, such as a thread, between a tooth and gingiva to push a gum away from the tooth in order to provide a clear view of a margin area, i.e., a boundary area between the tooth and the gingiva, when producing an impression or a prosthesis. For example, when a cord is inserted between the tooth and the gingiva, and the gum is pushed away from the tooth after a certain time period, the cord may be removed and then a scan may be performed.

Referring to FIG. 13, 1300A illustrates that the margin area, which is the boundary area between the tooth and the gingiva, is unclear. 1300B illustrates that the tooth may be stained with blood in an operation of inserting the cord into the margin area, which is the boundary area between the tooth and the gingiva. 1300C illustrates that the blood remains even when the cord is removed from the margin area. 1300D illustrates that, when the tooth is scanned after removing the cord, the blood on the tooth, particularly near the marginal area of the tooth, is also scanned, and thus, resulting scan data is unclear due to the blood.

As such, when the tooth are scanned after performing the cord packing operation, and foreign substances such as blood are on the scanned tooth, clear scan data cannot be obtained, and thus, scanning of the tooth needs to be performed again from the beginning to obtain data, which is inconvenient. In addition, because the tooth may be stained with blood again during cord packing, there is a possibility that blood will be included in scan data obtained through rescanning. In addition, there is a possibility that accurate margin area data will not be obtained, because such an operation as wiping up blood after a cord packing operation may change the state of the tooth and the gingiva pushed away from each other.

Thus, after obtaining scan data for the oral cavity including the margin area, only a partial area stained with foreign substances may be set as an attribute control area, and only the area set as the attribute control area may be rescanned to easily change the color of the attribute control area.

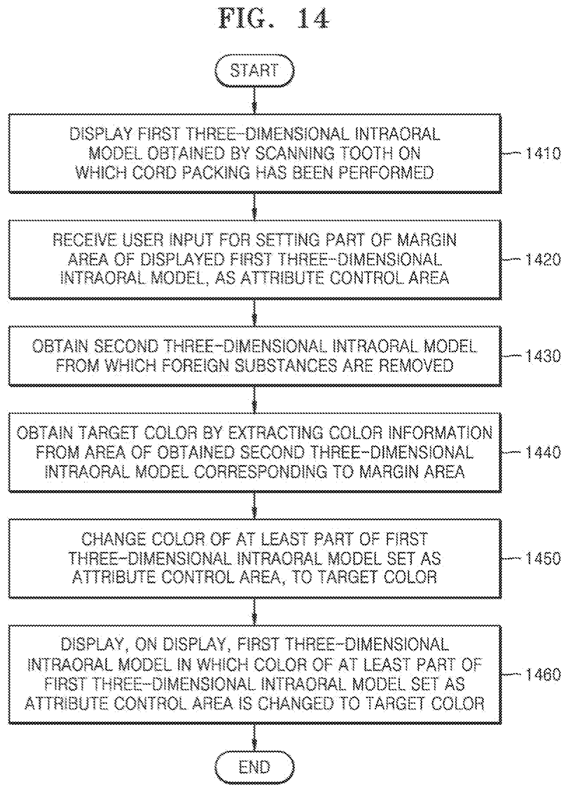

FIG. 14 is a flowchart of an example of an operating method using an attribute control area in a cord packing operation, according to an embodiment.

Referring to FIG. 14, in operation 1410, the data processing device 100 may display a first three-dimensional intraoral model obtained by scanning a tooth on which cord packing has been performed.

For example, the intraoral scanner may transmit, to the data processing device 100, two-dimensional image data obtained by scanning the tooth after performing a cord packing operation according to the method illustrated in FIG. 13, and the data processing device 100 may generate and display the first three-dimensional intraoral model based on the received two-dimensional image data.

In operation 1420, the data processing device 100 may receive a user input for setting a part of a margin area of the displayed first three-dimensional intraoral model, as an attribute control area.

In operation 1430, the data processing device 100 may obtain a second three-dimensional intraoral model from which foreign substances are removed.

For example, the intraoral scanner may transmit, to the data processing device 100, two-dimensional image data obtained by scanning the tooth in the area corresponding to the margin area set as the attribute control area after blood or saliva is removed the tooth, and the data processing device 100 may obtain the second three-dimensional intraoral model based on the two-dimensional image data.

In operation 1440, the data processing device 100 may obtain a target color by extracting color information from an area of the obtained second three-dimensional intraoral model corresponding to the margin area.

In operation 1450, the data processing device 100 may change the color of at least a part of the first three-dimensional intraoral model set as the attribute control area, to the target color.

In operation 1460, the data processing device 100 may display, on the display, the first three-dimensional intraoral model in which the color of the at least a part of the first three-dimensional intraoral model set as the attribute control area is changed to the target color.

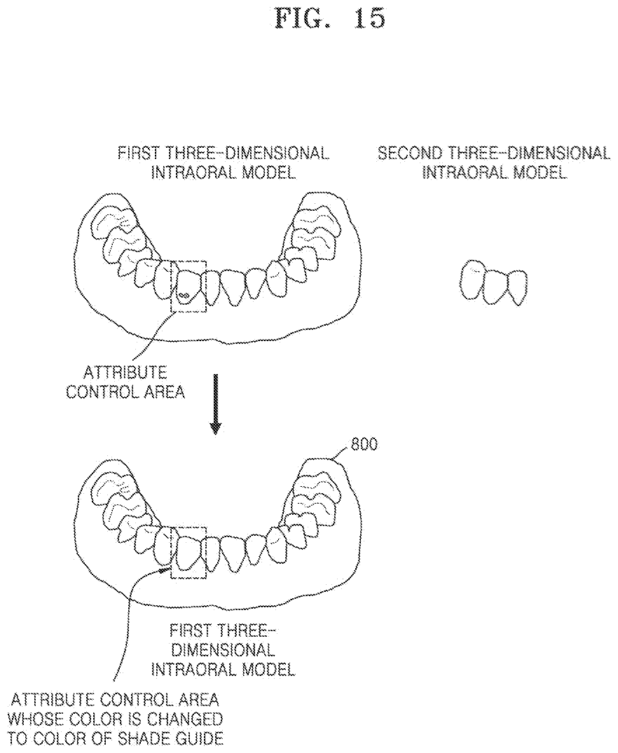

FIG. 15 is a diagram illustrating an example in which the color an attribute control area is changed to a target color, according to an embodiment.

Referring to FIG. 15, in the first three-dimensional intraoral model, foreign substances such as blood on a tooth due to a cord packing operation may be scanned, and thus may be expressed in scan data. Accordingly, the data processing device 100 may set an area in which the foreign substances are expressed, as an attribute control area.

In addition, the data processing device 100 may receive, from the intraoral scanner, scan data obtained by scanning an area corresponding to the attribute control area after the foreign substances are removed, and generate a second three-dimensional intraoral model based on the received scan data.

The data processing device 100 may identify an area of the second three-dimensional intraoral model corresponding to the area of the first three-dimensional intraoral model set as the attribute control area, extract color information of the identified area, and change the color of the attribute control area by using the extracted color information. Through this operation, a part expressed by the foreign substances in the attribute control area may be changed to a clear color.

According to an embodiment, when generating a three-dimensional intraoral model by using an impression, an attribute control area may be used.

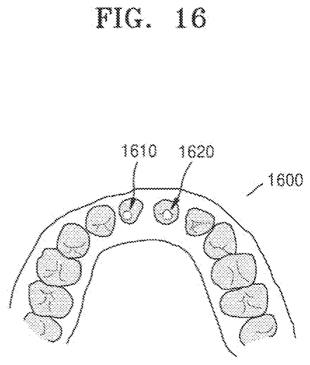

FIG. 16 is a diagram illustrating an example of a three-dimensional intraoral model obtained by scanning an oral cavity with teeth having holes thereon, according to an embodiment.

The intraoral scanner 51 may obtain surface data of a tooth of a patient by projecting light to the teeth by using a projector, and obtaining light reflected from the tooth by using an image sensor. However, when there is a hole on the tooth of the patient, it may be difficult for the light projected by the projector to accurately reach the surface of the hole on the tooth, and it may also be difficult for the image sensor to obtain the light reflected from the surface of the hole. Therefore, in a three-dimensional intraoral model 1600 generated based on data obtained by scanning a patient's teeth having holes thereon, it is difficult to obtain scan data for parts corresponding to the holes of the teeth, and thus, as illustrated in FIG. 16, parts 1620 and 1630 remaining as holes without filling the surface may be formed. Accordingly, a method of generating a three-dimensional intraoral model by using an impression in a case in which a tooth of a patient has a hole thereon has been proposed.

Impression (impression taking) refers to producing a negative imprint of the shape of teeth and oral tissues necessary for dental treatment such as tooth restoration or prosthetics, and a dental model may be produced by pouring gypsum or the like into a resulting impression mold and hardening it. Here, the obtained impression is a negative imprint of teeth and thus may relatively accurately express a part such as a hole on a tooth. Thus, two-dimensional image data obtained by scanning the impression with an intraoral scanner, the part corresponding to the hole on the tooth may be clearly expressed, and thus, data of the part corresponding to the hole on the tooth may be obtained more accurately than when directly scanning the oral cavity of the patient. However, data obtained by scanning an impression in this manner does not include color information of actual teeth. That is, because color information of the actual teeth is not included in the impression, color information of a material of the negative imprint of the teeth may be obtained but the actual color of the teeth cannot be obtained by scanning the impression. Thus, in this case, a color of the first three-dimensional intraoral model may be changed to the color of the actual teeth by setting, as an attribute control area, the entire area of the first three-dimensional intraoral model obtained by scanning the impression, and changing the color of the set attribute control area to a color of a second three-dimensional intraoral model obtained by scanning the actual teeth.

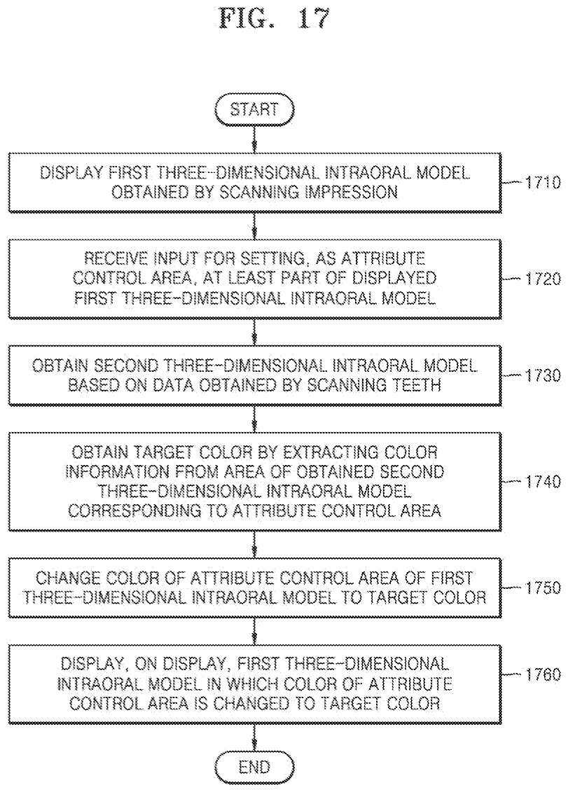

FIG. 17 is an example of an operation flowchart of a method of using an attribute control area for an impression scan, according to an embodiment.

Referring to FIG. 17, in operation 1710, the data processing device 100 may display a first three-dimensional intraoral model obtained by scanning the impression.

For example, when the intraoral scanner scans the impression that is a negative imprint for casting a dental model of teeth of a patient, and transmits two-dimensional image data obtained by the scanning to the data processing device 100, the data processing device 100 may generate and display a first three-dimensional intraoral model based on the received two-dimensional image data.

In operation 1720, the data processing device 100 may receive an input for setting, as an attribute control area, at least a part of the displayed first three-dimensional intraoral model.

According to an embodiment, the data processing device 100 may receive an input for setting, as an attribute control area, the entire area of the first three-dimensional intraoral model based on the impression scan.

In operation 1730, the data processing device 100 may obtain a second three-dimensional intraoral model based on data obtained by scanning actual teeth of the patient.

In operation 1740, the data processing device 100 may obtain a target color by extracting color information from an area of the obtained second three-dimensional intraoral model corresponding to the attribute control area. The target color may be the actual color of the teeth of the patient.

For example, the data processing device 100 may identify a position in the second three-dimensional intraoral model corresponding to the attribute control area by aligning the second three-dimensional intraoral model with the first three-dimensional intraoral model. For example, ICP or the like may be used for the alignment.

In operation 1750, the data processing device 100 may change the color of the attribute control area of the first three-dimensional intraoral model, to the target color.

In operation 1760, the data processing device 100 may display, on the display, the first three-dimensional intraoral model in which the color of the attribute control area is changed to the target color.

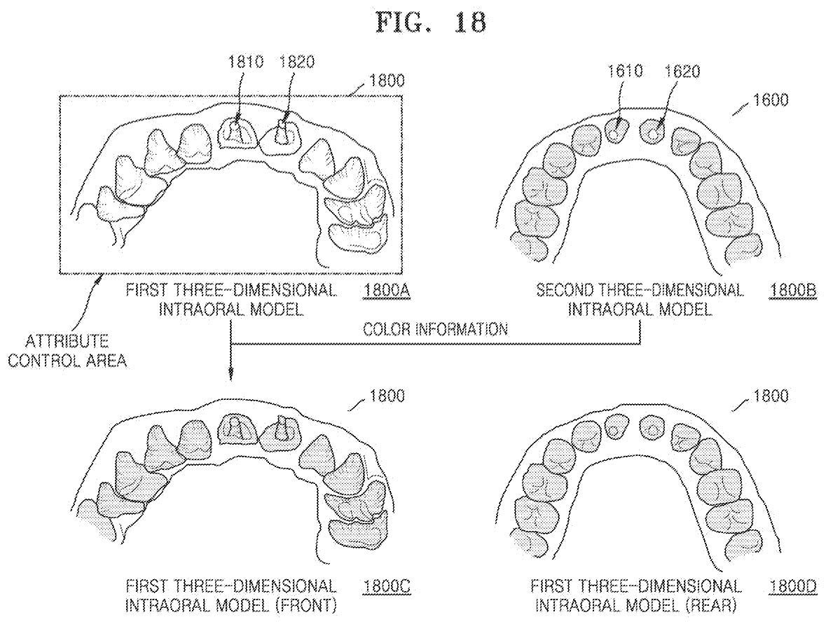

FIG. 18 is a diagram illustrating an example of changing the color of a tooth by setting an attribute control area for an impression scan, according to an embodiment.

Referring to FIG. 18, the data processing device 100 may obtain a first three-dimensional intraoral model 1800 based on scan data obtained by scanning a dental impression (1800A). In the dental impression, depressed parts, such as a hole on a tooth, are expressed as embossments 1810 and 1820, and thus, the first three-dimensional intraoral model 1800 based on the impression scan may accurately express the shape of a hole on a tooth by obtaining coordinate information of a depressed part such as a hole on a tooth. However, because the data is obtained by scanning the dental impression, color information of the first three-dimensional intraoral model 1800 is based on the color of the dental impression and does not reflect the actual color of the teeth. The data processing device 100 may set the entire area of the first three-dimensional intraoral model 1800 as an attribute control area in order to change the color of the first three-dimensional intraoral model 1800 to an actual tooth color.

The data processing device 100 may obtain the second three-dimensional intraoral model 1600 based on scan data obtained by scanning the teeth of the patient. It is difficult for a scanner to completely obtain information of parts corresponding to holes 1610 and 1620 on teeth, and thus, color information or position information of the parts may not be properly expressed (1800B).

The data processing device 100 may obtain the first three-dimensional intraoral model 1800 in which the color of the teeth is changed to the color of the actual teeth, by changing the color of the entire area of the first three-dimensional intraoral model 1800 set as the attribute control area by using the color of the second three-dimensional intraoral model 1600 (1800C). Here, color information corresponding to the parts corresponding to the holes 1610 and 1620 on the teeth may be obtained by using the color of an area surrounding the holes 1610 and 1620.

When the first three-dimensional intraoral model 1800 in which the color of the teeth is changed to the actual tooth color is turned upside down, the rear surface of the first three-dimensional intraoral model 1800 may correspond to a result of scanning the teeth of the patient (1800D).

A method of processing an intraoral image according to an embodiment of the present disclosure may be embodied as program instructions executable by various computer devices, and recorded on a computer-readable medium. In addition, an embodiment of the present disclosure may be implemented in a computer-readable recording medium having recorded thereon one or more programs including instructions for executing the method of processing an intraoral image.

The computer-readable recording medium may include program instructions, data files, data structures, or the like separately or in combinations. Here, examples of the computer-readable recording medium may include magnetic media such as hard disks, floppy disks, or magnetic tapes, optical media such as compact disc ROMs (CD-ROMs) or digital video discs (DVDs), magneto-optical media such as floptical disks, and hardware devices such as ROM, RAM, and flash memory, which are configured to store and execute program instructions.

Here, the machine-readable storage medium may be provided in the form of a non-transitory storage medium. Here, the ‘non-transitory storage medium’ may mean that the storage medium is a tangible device. In addition, the ‘non-transitory storage medium’ may include a buffer in which data is temporarily stored.

According to an embodiment, the method of processing an intraoral image according to various embodiments disclosed herein may be included in a computer program product and then provided. The computer program product may be distributed in the form of a machine-readable storage medium (e.g., a CD-ROM). Alternatively, the computer program product may be distributed (e.g., downloaded or uploaded) online through an application store (e.g., PlayStore™, etc.) or directly between two user devices (e.g., smart phones). In detail, the computer program product according to an embodiment of the present disclosure may include a storage medium having recorded thereon a program including at least one instruction for performing the method of processing an intraoral image according to an embodiment of the present disclosure.

Although embodiments have been described above in detail, the scope of the present disclosure is not limited thereto, and various modifications and alterations by those skill in the art using the basic concept of the present disclosure defined in the following claims also fall within the scope of the present disclosure.

1. A method of processing a three-dimensional intraoral model, the method comprising:
displaying a first three-dimensional intraoral model on a display;
receiving an input for setting at least a part of the displayed first three-dimensional intraoral model as an attribute control area;
obtaining a second three-dimensional intraoral model;
changing a color of the at least a part of the first three-dimensional intraoral model set as the attribute control area, to a target color representing a color of an area of the obtained second three-dimensional intraoral model corresponding to the attribute control area; and
displaying, on the display, the first three-dimensional intraoral model in which the color of the attribute control area is changed to the target color.

2. The method of claim 1, wherein the changing to the target color comprises identifying the area of the obtained second three-dimensional intraoral model corresponding to the attribute control area, and obtaining the target color by extracting color information from the identified area.

3. The method of claim 2, wherein the obtaining of the second three-dimensional intraoral model comprises obtaining the second three-dimensional intraoral model based on data obtained by scanning a tooth in a state in which an object is arranged to be overlaid on a front surface of the tooth.

4. The method of claim 3, wherein the object is arranged at a tooth position corresponding to a position set as the attribute control area in the three-dimensional

5. The method of claim 1, wherein the obtaining of the target color comprises identifying a position set as the attribute control area in the first three-dimensional intraoral model, and obtaining the target color by extracting color information from an area of the second three-dimensional intraoral model corresponding to the identified position.

6. The method of claim 1, wherein the receiving of the input for setting the at least a part of the displayed first three-dimensional intraoral model as the attribute control area comprises receiving an input for setting the attribute control area in the first three-dimensional intraoral model obtained by scanning a tooth on which cord packing has been performed.

7. The method of claim 6, wherein the changing of the color of the at least a part of the first three-dimensional intraoral model set as the attribute control area to the target color comprises obtaining the second three-dimensional intraoral model generated by scanning a tooth from which foreign substances are removed, obtaining the target color by extracting color information from the area of the obtained second three-dimensional intraoral model corresponding to the attribute control area, and changing the color of the at least a part of the first three-dimensional intraoral model set as the attribute control area to the obtained target color.

8. The method of claim 1, wherein the first three-dimensional intraoral model comprises a three-dimensional intraoral model obtained by scanning a dental impression.

9. The method of claim 8, wherein the changing of the color of the at least a part of the first three-dimensional intraoral model set as the attribute control area to the target color comprises obtaining the second three-dimensional intraoral model by scanning a tooth, obtaining the target color by extracting color information from the area of the obtained second three-dimensional intraoral model corresponding to the attribute control area, and changing the color of the at least a part of the first three-dimensional intraoral model set as the attribute control area to the obtained target color.

10. A device for processing a three-dimensional intraoral model, the device comprising:
a processor; and
a memory,
wherein the processor is configured to execute one or more instructions stored in the memory to display a first three-dimensional intraoral model on a display, receive an input for setting at least a part of the displayed first three-dimensional intraoral model as an attribute control area, obtain a second three-dimensional intraoral model, change a color of the at least a part of the first three-dimensional intraoral model set as the attribute control area, to a target color representing a color of an area of the obtained second three-dimensional intraoral model corresponding to the attribute control area, and display, on the display, the first three-dimensional intraoral model in which the color of the attribute control area is changed to the target color.

11. The device of claim 10, wherein the processor is further configured to execute the one or more instructions stored in the memory to identify the area of the obtained second three-dimensional intraoral model corresponding to the attribute control area, and obtain the target color by extracting color information from the identified area.

12. The device of claim 11, wherein the processor is further configured to execute the one or more instructions stored in the memory to obtain the second three-dimensional intraoral model based on data obtained by scanning a tooth in a state in which an object is arranged to be overlaid on a front surface of the tooth.

13. The device of claim 12, wherein the object is arranged at a tooth position corresponding to a position set as the attribute control area in the three-dimensional

14. The device of claim 10, wherein the processor is further configured to execute the one or more instructions stored in the memory to identify a position set as the attribute control area in the first three-dimensional intraoral model, and obtain the target color by extracting color information from an area of the second three-dimensional intraoral model corresponding to the identified position.

15. The device of claim 10, wherein the processor is further configured to execute the one or more instructions stored in the memory to receive an input for setting the attribute control area in the first three-dimensional intraoral model obtained by scanning a tooth on which cord packing has been performed.

16. The device of claim 15, wherein the processor is further configured to execute the one or more instructions stored in the memory to obtain the second three-dimensional intraoral model generated by scanning a tooth from which foreign substances are removed, obtain the target color by extracting color information from the area of the obtained second three-dimensional intraoral model corresponding to the attribute control area, and change the color of the at least a part of the first three-dimensional intraoral model set as the attribute control area to the obtained target color.

17. The device of claim 10, wherein the first three-dimensional intraoral model comprises a three-dimensional intraoral model obtained by scanning a dental impression.

18. The device of claim 17, wherein the processor is further configured to execute the one or more instructions stored in the memory to obtain the second three-dimensional intraoral model by scanning a tooth, obtain the target color by extracting color information from the area of the obtained second three-dimensional intraoral model corresponding to the attribute control area, and change the color of the at least a part of the first three-dimensional intraoral model set as the attribute control area to the obtained target color.

19. A computer-readable recording medium having recorded thereon a program for causing a computer to execute a method of processing a three-dimensional intraoral model, the method comprising:
displaying a first three-dimensional intraoral model on a display;
receiving an input for setting at least a part of the displayed first three-dimensional intraoral model as an attribute control area;
obtaining a second three-dimensional intraoral model;
changing a color of the at least a part of the first three-dimensional intraoral model set as the attribute control area, to a target color representing a color of an area of the obtained second three-dimensional intraoral model corresponding to the attribute control area; and
displaying, on the display, the first three-dimensional intraoral model in which the color of the attribute control area is changed to the target color.
